SEQUENCE LISTING (4 sequences)

SEQ ID NO: 1 - DNA, 431 bp, synthetic construct
  source 1..431: mol_type = other DNA
tgatacactctgcgacttttttggaattgggattgatgatctactggagtttgtccaaagctcaaataaatacagtgtcccaacaacgtggggttcatttaacacacccgagttctttgttgatctgaaaaaaatggttggagctaaagctcgacatactctattatctgtaacgtggaataattctcagcctgacgatacggccttttttgaagtcagcatttctccgttaagcaaaagcgatgctgaacaattctccggggaaggcaagccttccggttctgaattactagatggcaatctttttaaacgagactttaaaagacagacacgagatgttgctaaagaaacatcggaagttatattaagaatgcttattgaaagcgagttttttaaaaagcaggtttcttctatagtcggtgctaatgtga

SEQ ID NO: 2 - DNA, 18 bp, synthetic construct
  source 1..18: mol_type = other DNA
tgatacactctgcgactt

SEQ ID NO: 3 - DNA, 18 bp, synthetic construct
  source 1..18: mol_type = other DNA
tcacattagcaccgacta

SEQ ID NO: 4 - DNA, 1457 bp, synthetic construct
  source 1..1457: mol_type = other DNA
ctatctgcaagtcgaacgagttctgattattgaaaggtgcttgcatcttgatttaattttgaacgagtggcggacgggtgagtaacacgtgggtaacctgcccttaagtgggggataacatttggaaacagatgctaataccgcataaatccaagaaccgcatggttcttggctgaaagatggcgtaagctatcgcttttggatggacccgcggcgtattagctagttggtgaggtaacggctcaccaaggcaatgatacgtagccgaactgagaggttgatcggccacattgggactgagacacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagtgcatcggaaactggraaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcatttagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaacccttttagggagcgagccgtctaaaggggggaaa

(19) United States
(12) Patent Application Publication
(10) Pub. No.: US 2025/0312394 A1
(43) Pub. Date: Oct. 9, 2025

(54) Lacticaseibacillus Rhamnosus DY801 and Application Thereof

(71) Applicant: NANFANG HOSPITAL, SOUTHERN MEDICAL UNIVERSITY, Guangzhou (CN)

(72) Inventors: Yi Ding, Guangzhou (CN); Lu Yu, Guangzhou (CN); Zhenhui Chen, Guangzhou (CN); Bin Chen, Guangzhou (CN); Yaowei Zhang, Guangzhou (CN)

(21) Appl. No.: 18/627,441

(22) Filed: Apr. 4, 2024

(51) Int. Cl. A61K 35/747; A61P 1/00; C12N 1/20; C12Q 1/689; C12R 1/225

(52) U.S. Cl. CPC A61K 35/747; A61P 1/00; C12N 1/205; C12Q 1/689; C12R 2001/225

(57) ABSTRACT

Provided in the present disclosure is a strain of Lacticaseibacillus rhamnosus DY801 and an application thereof. The Lacticaseibacillus rhamnosus DY801 is deposited in the Guangdong Microbial Culture Collection Center (GDMCC) on Oct. 9, 2022, with a deposit number of GDMCC 62853. The Lacticaseibacillus rhamnosus DY801 provided by the present disclosure may inhibit oxidative stress in the intestinal tract and damage to the small intestinal villi under radiotherapy and chemotherapy conditions by producing a large amount of glutathione in order to reduce inflammatory cytokines, which is also tolerant to artificial gastric and intestinal fluids, and has no obvious toxic side effects on the human body, providing a high level of safety.

### REFERENCE TO SEQUENCE LISTING

This application includes a Sequence Listing filed electronically as an XML file named Sequence listing_SCHPY-24013-USPT.xml, created on Apr. 2, 2024, with a size of 6,092 bytes. The Sequence Listing is incorporated herein by reference.

### FIELD

The present disclosure relates to the technical field of microbiology and, particularly, to a strain of Lacticaseibacillus rhamnosus DY801 and an application thereof.

### BACKGROUND

Radiotherapy and chemotherapy are one of the common clinical therapies for the treatment of pelvic malignant tumors. Radiation intestinal injuries occur in approximately 75% to 81% of patients with pelvic malignant tumors after radiotherapy. Chemotherapy intestinal injuries occur in about 50% to 80% of patients after chemotherapy, especially chemotherapy regimens containing fluorouracil and irinotecan. In particular, the translocation of intestinal flora due to severe diarrhea leads to septicemia and even a 5% mortality rate. Radiotherapy and chemotherapy intestinal injuries that occur during treatment lead to a significant decrease in quality of life, poor treatment compliance, prolongation of the entire treatment regimen, and ultimately affect the overall survival prognosis of patients.

The current status of the treatment of radiotherapy and chemotherapy intestinal injuries is not encouraging. The treatment of radiotherapy intestinal injuries still remains at the stage of surgical resection of severely diseased intestinal segments and experimental drugs, and there is a dearth of consensus or guidelines for the diagnosis and treatment of these injuries. The treatment for chemotherapy intestinal injuries is mainly supportive therapy and drug therapy, in which the drug therapy is mainly based on such as loperamide, sulfasalazine and octreotide, but there are still 9-30% of patients ineffective for the above drug therapy. However, even drug therapy, led by NSAIDs, steroid hormone, and antibiotics, only improves local inflammation but not the intestinal flora disruption caused by the treatment, leading to poor overall treatment effects. Additionally, the drugs themselves may cause various adverse reactions, such as nausea and vomiting, which limits the widespread use of the drugs.

Lacticaseibacillus rhamnosus, a species of Lacticaseibacillus, is an anaerobic, acid-resistant, non-spore-forming, gram-positive probiotic bacteria present in the intestinal tract of humans and animals. Lacticaseibacillus rhamnosus is able to survive and proliferate in gastric acidic environments and bile-containing culture medium, with the ability to adhere to intestinal epithelial cells. Additionally, Lacticaseibacillus rhamnosus generates biofilms, promotes intestinal crypt survival, reduces apoptosis of intestinal epithelial cells and produces a range of soluble factors beneficial to the intestinal tract. Lacticaseibacillus rhamnosus also suppresses some pathogens such as Salmonella, Escherichia coli, and highly pathogenic Staphylococcus aureus. Lacticaseibacillus rhamnosus also possesses potent immunomodulatory properties, as evidenced by the ability to reduce monocyte activation and expression of inflammatory cytokines, and may also enhance macrophage function. Also, Lacticaseibacillus rhamnosus possesses the function of treating antibiotic-associated diarrhea, necrotizing enterocolitis in newborns, Clostridium difficile-induced diarrhea and other inflammatory disorders of the digestive system, which has become one of the Lactobacilli widely used internationally and domestically. However, the existing Lacticaseibacillus rhamnosus is poorly tolerated by human gastric and intestinal fluids and produces low content of glutathione, resulting in a limited role of Lacticaseibacillus rhamnosus in the gastrointestinal tract of the host, which then affects the therapeutic efficacy of Lacticaseibacillus rhamnosus. Therefore, the search for a Lacticaseibacillus rhamnosus that is tolerant to host gastric and intestinal fluids is of paramount importance.

### SUMMARY

The present disclosure provides a strain of Lacticaseibacillus rhamnosus DY801 and an application thereof, in which the strain of Lacticaseibacillus rhamnosus enables to inhibit oxidative stress in the intestinal tract by producing a large amount of glutathione and is tolerant to gastric and intestinal fluids of the human body, which allows it to be used for the prevention and/or treatment of radiotherapy and chemotherapy intestinal injuries.

In accordance with a first aspect of the present disclosure, provided is a strain of Lacticaseibacillus rhamnosus DY801, in which the strain of Lacticaseibacillus rhamnosus is deposited on Oct. 9, 2022 in the Guangdong Microbial Culture Collection Center located at Guangdong Institute of Microbiology, No. 100, Xianlie Central Road, Guangzhou, Guangdong Province, China, with a deposit number of GDMCC 62853.

In the present disclosure, a new strain of Lacticaseibacillus rhamnosus with the taxonomy designation Lacticaseibacillus rhamnosus DY801 is isolated from fresh feces of healthy adults originating from Guangzhou, Guangdong Province, China. Compared to existing Lacticaseibacillus rhamnosus, the Lacticaseibacillus rhamnosus DY801 provided by the present disclosure is a superior strain of indigenous origin, as evidenced by the fact that the strain thereof produces bacterial culture products, such as glutathione, which inhibits oxidative stress and inflammation in the intestinal tract. The strain thereof is tolerant to artificial gastric and intestinal fluids, with high survival rates in the stomach and intestines, and is able to colonize the intestinal tract and restore intestinal flora homeostasis. Moreover, the strain thereof reduces damage to small intestinal villi under radiotherapy and chemotherapy conditions, decreases inflammatory cytokines, and accelerates intestinal tissue repair. Additionally, compared with conventional drugs for treating radiotherapy intestinal injuries, the Lacticaseibacillus rhamnosus DY801 provided by the present disclosure has no obvious toxic effects on the human body, providing a high level of safety. In summary, the Lacticaseibacillus rhamnosus DY801, with many excellent properties, may be used for prevention and treatment of digestive disorders caused by radiotherapy and chemotherapy intestinal injuries, which solves the problems in the prior art of inability to effectively treat the radiotherapy and chemotherapy intestinal injuries occurring after radiotherapy and chemotherapy for pelvic malignant tumors, which offers great application prospects in the preparation of drugs for the prevention and/or treatment of radiotherapy and chemotherapy intestinal injuries or food and health care products with auxiliary protection against radiation hazards.

In accordance with a second aspect of the present disclosure, provided is the application of the aforementioned Lacticaseibacillus rhamnosus DY801 in the preparation of drugs for the prevention and/or treatment of digestive disorders.

Preferably, the digestive disorders mentioned above include at least one of abdominal pain, bloating, nausea, vomiting, diarrhea, and constipation.

Preferably, the digestive disorders mentioned above are caused by intestinal injuries.

Preferably, the intestinal injuries mentioned above are radiotherapy or chemotherapy intestinal injuries.

In accordance with a third aspect of the present disclosure, provided is a drug for prevention and/or treatment of digestive disorders, the drug including the aforementioned Lacticaseibacillus rhamnosus DY801 or cultures thereof.

The Lacticaseibacillus rhamnosus DY801 or cultures thereof provided by the present disclosure is applied in a preparation of drugs for prevention and/or treatment of digestive disorders caused by radiotherapy and chemotherapy intestinal injuries, and the drug obtained from the preparation is effective for prevention and/or treatment of radiotherapy and chemotherapy intestinal injuries, which solves the problems in the prior art of inability to effectively treat the radiotherapy and chemotherapy intestinal injuries occurring after radiotherapy and chemotherapy for pelvic malignant tumors.

Preferably, the digestive disorders mentioned above include at least one of abdominal pain, bloating, nausea, vomiting, diarrhea, and constipation.

Preferably, the digestive disorders mentioned above are caused by intestinal injuries.

Preferably, the intestinal injuries mentioned above are radiotherapy or chemotherapy intestinal injuries.

Preferably, the drug mentioned above further includes a pharmaceutically acceptable excipient, the pharmaceutically acceptable excipient including at least one of stabilizers, wetting agents, emulsifiers, binders, and isotonic agents.

Preferably, the drug mentioned above is presented in at least one of tablets, granules, dispersions, capsules, solutions, suspensions, and lyophilized dosage forms.

In accordance with a fourth aspect of the present disclosure, provided is an application of the Lacticaseibacillus rhamnosus DY801 mentioned above in a preparation of a food or healthcare product having auxiliary protection against radiation hazards.

In accordance with a fifth aspect of the present disclosure, provided is a food product having auxiliary protection against radiation hazards, the food product including the Lacticaseibacillus rhamnosus DY801 mentioned above or cultures thereof.

The Lacticaseibacillus rhamnosus DY801 or cultures thereof provided by the present disclosure are applied in the preparation of food or healthcare products having auxiliary protection against radiation hazards, and the prepared food or healthcare products may provide certain auxiliary protection against radiation hazards.

In accordance with a sixth aspect of the present disclosure, provided is a nucleotide sequence that specifically recognizes the Lacticaseibacillus rhamnosus DY801 mentioned above, with the nucleotide sequence shown in SEQ ID: 1.

The nucleotide sequences provided in the present solution allow for effective differentiation of Lacticaseibacillus rhamnosus DY801 provided by the present disclosure from other isolated strains of Lacticaseibacillus rhamnosus.

In accordance with a seventh aspect of the present disclosure, provided is a primer set that specifically recognizes the Lacticaseibacillus rhamnosus DY801 mentioned above, the primer set including a nucleotide sequence as shown in SEQ ID: 2 and SEQ ID: 3.

The primer set provided in the present solution enables the PCR reaction to amplify the DNA of the bacterium to be tested. If a product of 431 bp is amplified, it indicates that the bacterium to be tested is Lacticaseibacillus rhamnosus DY801, and if a product of 431 bp is not amplified, it indicates that the bacterium to be tested is not Lacticaseibacillus rhamnosus DY801.

In accordance with an eighth aspect of the present disclosure, provided is a method for identification of the Lacticaseibacillus rhamnosus DY801 mentioned above, in which the primer set mentioned above is used as a specific amplification primer, and the genomic DNA of the Lacticaseibacillus rhamnosus to be tested is used as a template for PCR amplification to obtain a PCR product, and sequencing or electrophoresis is utilized for identification of the PCR product.

### BRIEF DESCRIPTION OF THE DRAWINGS

FIG. 1 shows the colony morphology of Lacticaseibacillus rhamnosus DY801 obtained by isolation and cultivation in Example 1.

FIG. 2 shows a diagram of the results of bacteriophage Gram staining of Lacticaseibacillus rhamnosus DY801 obtained by isolation and cultivation in Example 1.

FIG. 3 shows a graph of the results of sequence matching of the 16S rDNA gene of Lacticaseibacillus rhamnosus DY801 in Example 2.

FIG. 4 shows a graph of the results of the glucose fermentation biochemical test for the identification of Lacticaseibacillus rhamnosus DY801 in Example 2.

FIG. 5 shows the whole genome sequencing map and genome circle diagram for Lacticaseibacillus rhamnosus DY801 in Example 3.

FIG. 6 shows a graph of the gross results of vital organs for the in vivo safety assessment in mice gavaged with Lacticaseibacillus rhamnosus DY801 in Example 4.

FIG. 7 shows a graph of the results of histopathological staining analysis of vital organs for in vivo safety assessment in mice gavaged with Lacticaseibacillus rhamnosus DY801 in Example 4.

FIG. 8 shows a graph of the detection results of the glutathione content produced by Lacticaseibacillus rhamnosus DY801 using liquid chromatography-mass spectrometry in Example 7.

FIG. 9 shows a survival curve after treating mice suffering from radiotherapy and chemotherapy intestinal injuries with Lacticaseibacillus rhamnosus DY801 in Example 8.

FIG. 10 shows an HE-stained image (40×) of intestinal tissue of mice suffering from radiotherapy and chemotherapy intestinal injuries after treatment with Lacticaseibacillus rhamnosus DY801 in Example 8.

FIG. 11 shows a graph of the detection results of blood inflammation cytokines in mice after treating mice suffering from radiotherapy and chemotherapy intestinal injuries with Lacticaseibacillus rhamnosus DY801 in Example 8.

### DETAILED DESCRIPTION OF THE EMBODIMENTS

The technical features of the technical solutions in the present disclosure are clearly and completely described below in conjunction with the specific implementations. Obviously, the examples described herein are only some of the examples of the present disclosure but not all of them. Based on the embodiments in the present disclosure, all other embodiments obtained by those skilled in the art without creative efforts fall within the scope of protection of the present disclosure.

### Example 1 Isolation of Lacticaseibacillus rhamnosus DY801

In the present example, a new strain of Lacticaseibacillus rhamnosus was isolated from fresh feces of healthy adults originating from Guangzhou, Guangdong Province, China. The Lacticaseibacillus was identified as Lacticaseibacillus rhamnosus DY801 using morphological features, culture characteristics and physiological and biochemical traits and genetic characterization 16S rDNA. The strain of Lacticaseibacillus rhamnosus was deposited on Oct. 9, 2022 in the Guangdong Microbial Culture Collection Center located at Guangdong Institute of Microbiology, No. 100, Xianlie Central Road, Guangzhou, Guangdong Province, China, with the taxonomy designation Lacticaseibacillus rhamnosus DY801, with a deposit number of GDMCC 62853.

In the present example, after collecting fresh feces from healthy adults originating from Guangzhou, Guangdong Province, China, a PBS buffer was added to the fresh feces for dilution at a solid-liquid ratio of 1:1000 (g/mL). 5 μL of the diluted fresh fecal samples mentioned above was inoculated into MRS culture medium and cultured anaerobically at a constant temperature of 37° C. for 48 hours, and then single colonies were picked and inoculated into MRS liquid culture medium for bacterial enrichment.

The MRS culture medium involved in the above culture process was formulated as follows: 10 g of peptone, 5 g of yeast extract, 10 g of beef extract, 20 g of glucose, 5 g of sodium acetate, 2 g of diammonium citrate, 1 mL of tween-80, 0.58 g of magnesium sulphate, 0.05 g of manganese sulphate, 2 g of dipotassium hydrogenphosphate, 15-17 g of agar, and 1,000 mL of water, with the pH adjusted to 6.12-6.2.

The morphological characteristics of Lacticaseibacillus rhamnosus DY801 obtained after isolation and cultivation of the present example were as follows:

### (1) Colony Characteristics

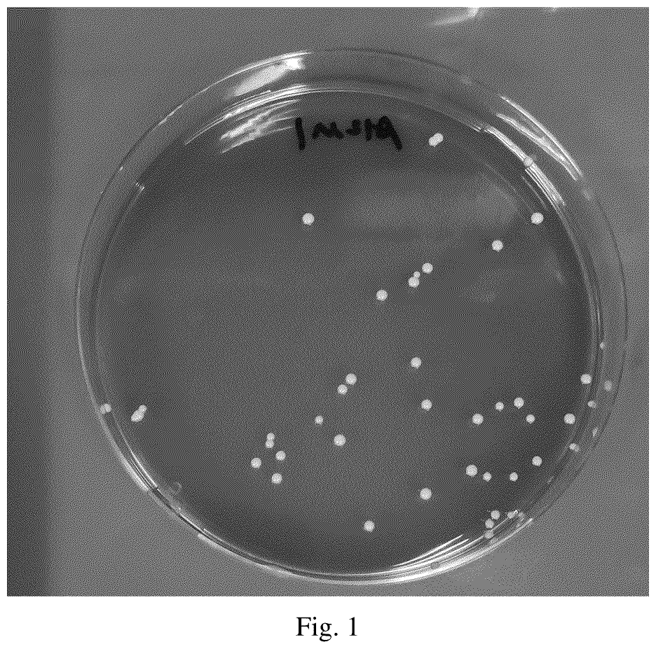

In the present example, Lacticaseibacillus rhamnosus DY801, which was obtained after isolation and cultivation, was isolated by scribing on a plate and cultured anaerobically at a constant temperature of 37° C. for 72 hours. The colony morphology thereof is shown in FIG. 1. As shown in FIG. 1, Lacticaseibacillus rhamnosus DY801 was in good growth condition, with raised, rounded colonies, smooth, fine, white-colored surface and neat edges.

### (2) Bacterial Characteristics

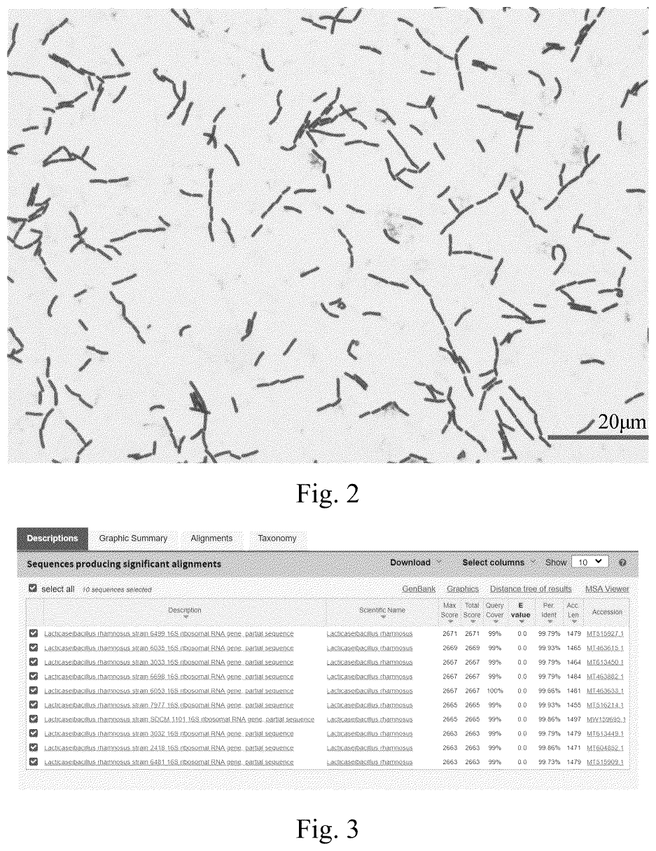

In the present example, the bacterial morphology of Lacticaseibacillus rhamnosus DY801, which was obtained after isolation and cultivation, was observed and also stained using Gram staining. The result is shown in FIG. 2. As shown in FIG. 2, Lacticaseibacillus rhamnosus DY801 was elongated rod-shaped, non-motile, non-budding, parthenogenetic anaerobic, and was Gram-stain positive.

### Example 2 Identification of Lacticaseibacillus rhamnosus DY801

In the present example, the total bacterial DNA of Lacticaseibacillus rhamnosus DY801 obtained by isolation and cultivation in Example 1 was extracted using a TIANamp Bacteria DNA Kit, and the steps of the extraction method were carried out according to the specification of the kit. PCR amplification of extracted DNA was performed using universal primers for 16S rDNA.

The nucleotide sequences of the universal primer pairs for 16S rDNA were:
forward primer 27f: 5′-AGAGTTTGATCCTGGCTCAG-3′,
reverse primer 1492r: 5′-GGTTACCTTGTTACGACTT-3′, and
the primers were synthesized by Beijing Tsingke Biotech Co., Ltd.

The PCR amplification reaction system was 20 μL in total, with 2 μL of template, 10 μL of TaKaRa Premix Taq™, 1 μL each of forward primer and reverse primer, and 6 μL of double-distilled water. A negative control was also set up. In the negative control reaction system, the template was replaced by double-distilled water, and the rest of the components were the same.

PCR amplification reaction conditions: 94° C. for 5 min; 94° C. for 60 s, 60° C. for 60 s, 72° C. for 90 s, for 30 cycles; 72° C. for 10 min; and stored at 4° C.

After PCR electrophoresis, the gel was cut, and the target strip gel was extracted and sequenced (nucleotide sequence as shown in SEQ ID: 4) by Sangon Biotech (Shanghai) Co., Ltd. The BLAST software tool was applied to match the 16S rDNA gene sequence of Lacticaseibacillus rhamnosus DY801 in the NCBI database of USA, and the results are shown in FIG. 3.

As shown by the sequencing results in FIG. 3, the 16S rDNA sequence homology between Lacticaseibacillus rhamnosus DY801 provided by the present disclosure and Lacticaseibacillus rhamnosus reaches 99%, identifying Lacticaseibacillus rhamnosus DY801 as Lacticaseibacillus rhamnosus, and designating it as DY801, i.e., Lacticaseibacillus rhamnosus DY801, which is obtained by the present disclosure after isolation and cultivation.

Additionally, in the present example, Lacticaseibacillus rhamnosus DY801 was further identified using a glucose fermentation test:

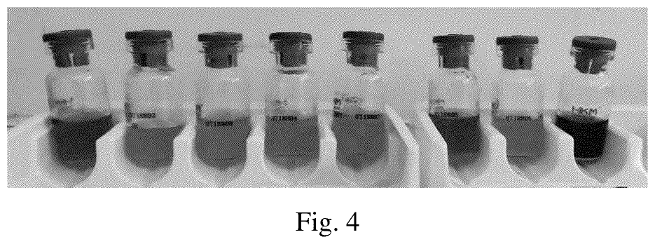

The biochemical metabolites of the isolated strain DY801 to be selected were detected according to the specifications of the novel microbial trace-biochemical series identification tube, and the results are shown in FIG. 4 and Table 1. In conjunction with data from the Bergey's Manual of Systematic Bacteriology as well as FIG. 4 and Table 1, it is evident that the physiological and biochemical characteristics of the strain thereof are basically consistent with those of the standard strain of Lacticaseibacillus rhamnosus ATCC53103.

TABLE 1
Biochemical identification of Lacticaseibacillus rhamnosus DY801

| Biochemical tube | | |
|---|---|---|
| taxonomy | ATCC53103 | DY801 |
| cellobiose | + | + |
| maltose | + | + |
| raffinose | + | + |
| mannitol | + | + |
| fructose | + | + |
| salicin | − | − |
| sorbitol | + | + |
| esculin hydrate | − | − |

Note:
“+” indicates a positive reaction, “−” indicates a negative reaction.

### Example 3 Whole Genome Sequencing of Lacticaseibacillus rhamnosus DY801

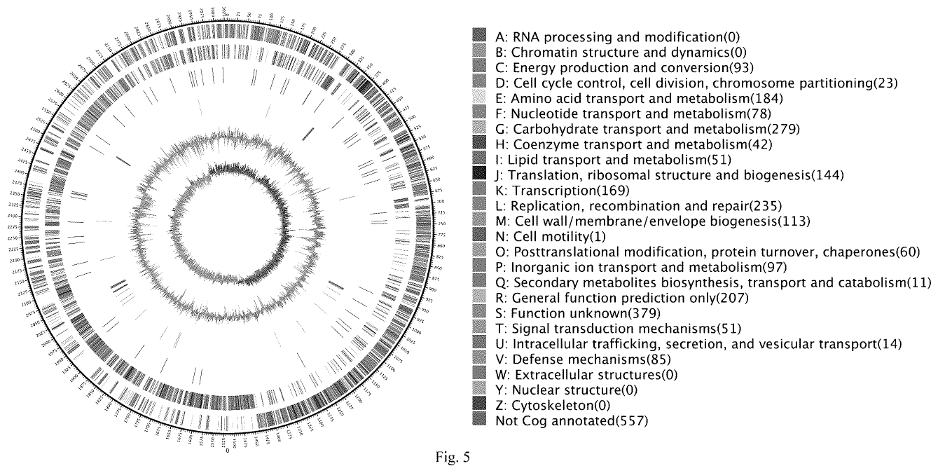

The genomic DNA of Lacticaseibacillus rhamnosus DY801 obtained by isolation and cultivation in Example 1 was extracted and quality checked for purity, concentration and integrity using Nanodrop, Qubit and 0.5% agarose gel electrophoresis. Also, the BluePippin fully automated nucleic acid recovery system was utilized to recover large fragments of DNA, the SQK-LSK109 ligation kit was used for library construction, on-line sequencing, and quality control was performed on the raw data after going off-line to filter low-quality and too-short-length reads. Subsequently, genome assembly was performed and the filtered reads were assembled from the beginning and the assembled draft genomes were corrected for errors. Then, genomic component analysis and genomic functional annotation, including PHI-base, CARD, and TCDB database annotation, were performed. In addition, genomic analysis and genome mapping were performed. The whole genome sequencing results of Lacticaseibacillus rhamnosus DY801 are shown in FIG. 5.

As shown in the genome sequencing map and genome circle diagram of FIG. 5, the genome size of Lacticaseibacillus rhamnosus DY801 is 3.01 Mb, with a GC ratio of 46.78%, and the genome contains 2,873 CDS regions, 3,626 bp of repetitive sequences, 60 tRNAs and 15 rRNAs. Genomic functional annotation suggests that Lacticaseibacillus rhamnosus DY801 contains two potential resistance genes i.e., poxtA and lmrB, yet does not contain virulence genes acquired by horizontal gene transfer.

### Example 4 Safety Assessment of Lacticaseibacillus rhamnosus DY801

The Lacticaseibacillus rhamnosus DY801 provided by the present disclosure was a probiotic isolated from fresh feces of healthy adults to ensure the safety and efficacy derived from the bacterial source. Additionally, the Lacticaseibacillus rhamnosus DY801 provided by the present disclosure, after colonization and reproduction in the human intestinal environment, merely adhered to the host's intestinal epithelial cells, and was able to become a layer of biological barriers of the intestinal mucosa, enhancing the host's intestinal mucosal barrier ability, and may act on the human body in the form of a viable bacterium directly to ensure the safety.

In the present example, the sensitivity of Lacticaseibacillus rhamnosus DY801 against eight antibiotics was tested using the broth trace-dilution method according to “the General Standard of Probiotics for Food Use” (hereinafter referred as General Standard) published by the Chinese Institute of Food Science and Technology. The eight antibiotics were: tetracycline, streptomycin, ciprofloxacin hydrochloride, clindamycin, vancomycin, chloramphenicol, ampicillin and gentamicin. The suspension of Lacticaseibacillus grown to logarithmic growth phase was adjusted to 1×10⁸ CFU/mL, followed by the addition of different concentrations of antibiotic diluents (from 1 to 64 mg/mL), and cultured anaerobically at 37° C. for 48 hours. The minimum inhibitory concentration (hereinafter referred as MIC) of Lacticaseibacillus rhamnosus DY801 for each antibiotic was read after 48 hours. The strain thereof was determined to be sensitive(S), intermediate (I), resistant (R) and not required (n.r.) for the antibiotic according to the bacterial resistance criteria provided in the General Standard, and the results are shown in Table 2.

TABLE 2
Drug sensitivity of Lacticaseibacillus rhamnosus
DY801 against different antibiotics

| | MIC in the | | |
|---|---|---|---|
| Types of | General Standard | Detected MIC | Determination of |
| antibiotics | (mg/L) | (mg/L) | the reading |
| tetracycline | 8 | 8 | S |
| streptomycin | 32 | 32 | S |
| ciprofloxacin | n.r | 64 | n.r. |
| hydrochloride | | | |
| clindamycin | 4 | 4 | S |
| vancomycin | n.r. | 64 | n.r. |
| chloromycetin | 4 | 2 | S |
| ampicillin | 4 | 4 | S |
| gentamycin | 16 | 16 | S |

As shown in Table 2, the MICs of Lacticaseibacillus rhamnosus DY801 against tetracycline, streptomycin, ciprofloxacin hydrochloride, clindamycin, vancomycin, chloramphenicol, ampicillin, and gentamicin are, in the following order, 8 mg/L, 32 mg/L, 64 mg/L, 4 mg/L, 64 mg/L, 2 mg/L, 4 mg/L, and 16 mg/L, which indicates that the strain thereof is sensitive to six antibiotic bacteria as specified by the General Standard.

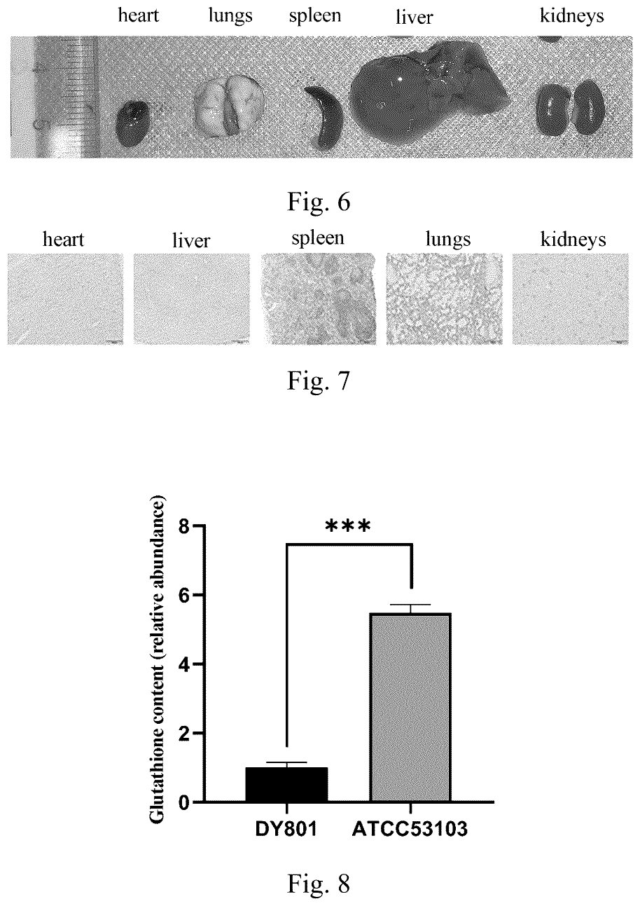

Additionally, in order to further evaluate the safety of Lacticaseibacillus rhamnosus DY801, three 6 to 8-week-old C57BL/6 mice were selected for experiments in the present example. Animals were acclimatized and fed in the animal room for 5 days prior to the experiment. The experimental animals and the experimental animal room complied with the national regulations, standardized compound feed was used, and the diet and water were not restricted. Mice were gavaged with Lacticaseibacillus rhamnosus DY801, and 0.2 mL of DY801 bacterial solution with an absorbance of OD₆₀₀=1 is gavaged daily. At the end of feeding, the experimental animals were executed by neck-breaking, the organs were dissected and removed by scalpel, the vital organs of the mice were observed, and the results are shown in FIG. 6. Also, histopathological staining analysis was carried out, and the results are shown in FIG. 7.

As shown in FIG. 6, no abnormalities are observed in the gross structure of the vital organs of the heart, liver, spleen, lungs and kidneys of mice. As shown in FIG. 7, histopathological staining of the mice does not show any tissue damage. The above results indicate that Lacticaseibacillus rhamnosus DY801 provided by the present disclosure is safe for in vivo application in mice.

### Example 5 Lacticaseibacillus rhamnosus DY801 has Good Tolerance to Artificial Gastric Fluid

Artificial gastric fluids with pH of 2.0, 3.0, and 4.0 were prepared separately. Lacticaseibacillus rhamnosus DY801, isolated and cultured in Example 1, was taken and diluted to a suspension of 109 CFU/mL with sterile PBS buffer. 1 μL of bacterial suspension was aspirated into 99 μL of artificial gastric fluids of different pH values and cultured in 96-well plates, and cultured anaerobically at 37° C. for 1˜3 h, and the initial and post-cultivation number of viable bacteria was assayed using a microplate reader. The results are shown in Table 3.

As shown in Table 3, compared to the standard strain of Lacticaseibacillus rhamnosus ATCC53103, Lacticaseibacillus rhamnosus DY801 provided by the present disclosure performs a better tolerance to artificial gastric fluids.

TABLE 3
Comparison of the performance of DY801 and ATCC53103
in tolerating artificial gastric fluid

| | Number of live bacteria (×10⁹ CFU/mL) | | |
|---|---|---|---|
| strains | pH = 2 | pH = 3 | pH = 4 |
| ATCC53103 | 15.81 ± 1.33 | 71.10 ± 5.22 | 77.85 ± 3.40 |
| DY801 | 37.69 ± 8.77 | 73.11 ± 8.00 | 84.58 ± 2.29 |

### Example 6 Lacticaseibacillus rhamnosus DY801 Performs Good Tolerance to Artificial Intestinal Fluids

An artificial intestinal fluid containing bile salts with a pH of 6.8 was prepared. Lacticaseibacillus rhamnosus DY801, isolated and cultured in Example 1, was taken and diluted to a bacterial suspension of 109 CFU/mL with sterile PBS buffer. 1 μL of bacterial suspension was aspirated into 99 μL of artificial intestinal fluids of different pH values and cultured in 96-well plates, and cultured anaerobically at 37° C. for 1˜3 h, and the initial and post-cultivation number of viable bacteria was assayed using a microplate reader. The results are shown in Table 4.

TABLE 4
Comparison of the performance of DY801 and ATCC53103
in tolerating artificial intestinal fluids

| | Number of viable bacteria (×10⁹ |
|---|---|
| | CFU/mL) |
| Strains | pH = 6.8 |
| ATCC53103 | 95.07 ± 1.09 |
| DY801 | 99.24 ± 1.90 |

As shown in Table 4, Lacticaseibacillus rhamnosus DY801 performs better resistance to artificial intestinal fluid compared to the standard strain of Lacticaseibacillus rhamnosus ATCC53103.

### Example 7 Lacticaseibacillus rhamnosus DY801 Performs Strong Glutathione Production Capacity

100 μL of Lacticaseibacillus rhamnosus DY801 bacterial solution with an absorbance adjusted to OD600=1 was added to 10 mL of MRS broth culture medium containing L-tryptophan and cultured for 48 hours. 0.5 mL of the bacterial suspension was taken into a 1.5 mL centrifuge tube, 205 μL of precipitant (acetonitrile:methanol=1:1) containing 5 μL of mixed internal standard was added, mixed by vortexing, and placed on ice for 30 minutes. The sample was centrifuged at 12,000 rpm for 10 minutes at 4° C., and the supernatant was taken in a sample vial as a sample to be tested for mass spectrometry analysis by liquid chromatography-mass spectrometry.

Preparation of blank sample: 5 μL of internal standard (IS), 100 μL of acetonitrile, and 100 μL of methanol were added into 500 μL of saline, mixed by vortexing, and placed on ice for 30 minutes. Then, the sample was centrifuged at 12,000 rpm for 10 minutes at 4° C., and the supernatant was taken into a sample vial. The mass spectrometry analysis was performed as above.

Preparation of quality control samples: 5 μL of internal standard (IS), 5 μL of STD (standard), 100 μL of acetonitrile, 100 μL of methanol were added to 500 μL of saline, mixed by vortexing, and placed on ice for 30 minutes. Then, the sample was centrifuged at 12,000 rpm for 10 minutes at 4° C., and the supernatant was taken into a sample vial. The mass spectrometry analysis was performed as above.

The glutathione content in the supernatant was assayed using liquid chromatography-mass spectrometry, i.e., the glutathione content in the sample to be tested was assayed, and the results are shown in FIG. 8.

As shown in FIG. 8, compared to the standard strain of Lacticaseibacillus rhamnosus ATCC53103, Lacticaseibacillus rhamnosus DY801 provided by the present disclosure performed a better production capacity of glutathione and a higher total amount of glutathione.

### Example 8 Lacticaseibacillus rhamnosus DY801 Reduces Mucosal Congestion and Edema, Leading to Effective Treatment of Radiotherapy and Chemotherapy Intestinal Injuries

24 C57BL/6 mice at 5-6 weeks of age were selected and randomly divided into 4 groups after 7 days of normal feeding: Control group (n=6), RCT group (n=6), RCT+ATCC53103 group (n=6), and RCT+DY801 group (n=6), in which RCT indicates that whole abdominal irradiation was performed.

From day 1 to 7, mice in the Control group were gavaged with 0.2 mL of PBS buffer, and mice in the RCT+ATCC53103 group and RCT+DY801 group were gavaged with 0.2 mL of ATCC53103 and DY801 bacterial solution with an absorbance of OD₆₀₀=1, respectively.

On day 8, after the mice in the RCT group, RCT+ATCC53103 group and RCT+DY801 group were fasted with food and water for 2 hours, the mice were anesthetized and irradiated using 6 MeV X-rays in the whole abdomen, in which the irradiation dose was 6Gy, followed by chemotherapy treatment, which was administered as a single intraperitoneal injection of 0.2 mL of 150 mg/Kg Fluorouracil injection, and the fasting and water fasting was lifted after the completion of the injection.

From day 9 to 11, mice in the Control group were gavaged with 0.2 mL of PBS buffer, and mice in the RCT+ATCC53103 group and RCT+DY801 group were gavaged with 0.2 mL of ATCC53103 and DY801 bacterial solutions with an absorbance of OD₆₀₀=1, respectively. Mice were dissected on day 30 to test for various cytokines.

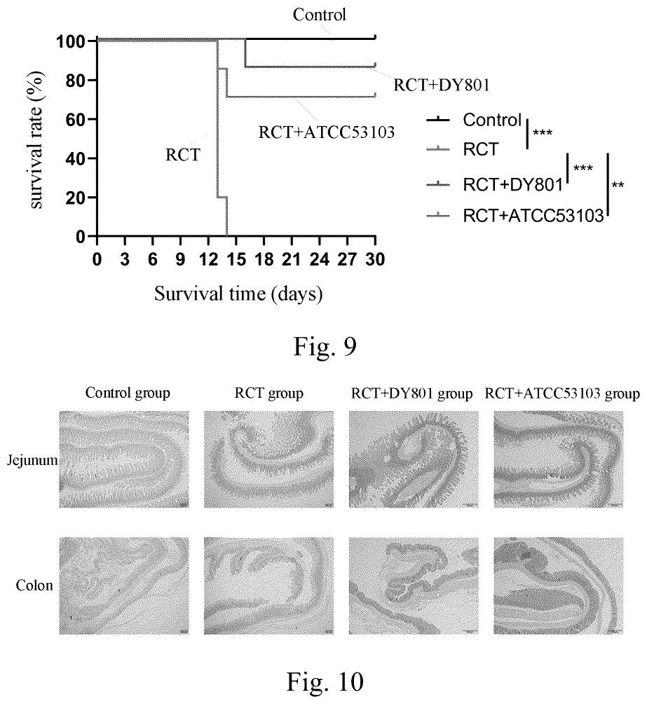

The survival time of mice in each group was analyzed by Kaplan-Meier survival analysis, and the results are shown in FIG. 9. As shown in FIG. 9, all mice in the Control group survive, all mice in the RCT group died before the end of the experiment, all mice in the RCT+ATCC53103 group and the RCT+DY801 group survived at the end of the experiment, and the number of mice in the RCT+DY801 group survived in greater numbers and for a longer period of time compared with that of the RCT+ATCC53103 group, in which the survival rate of mice in the RCT+DY801 group reached 83.3%. The above results indicate that Lacticaseibacillus rhamnosus DY801 provided by the present disclosure improves survival significantly in mice suffering from radiotherapy and chemotherapy intestinal injuries and the survival rate thereof is higher than that of the standard strain ATCC53103.

The results of staining (HE staining) using hematoxylin-eosin on the groups of mice treated with the above treatments are shown in FIG. 10. As shown in FIG. 10, intestinal histopathological damage such as destruction of glandular tissue, mucosal congestion and edema, and shortening of small intestinal villi occurred in the RCT group, whereas glandular structural damage was reduced and mucosal congestion and edema were significantly improved in the RCT+DY801 group. The above results indicate that the Lacticaseibacillus rhamnosus DY801 provided by the present disclosure reduces mucosal congestion and edema, leading to effective treatment of radiotherapy and chemotherapy intestinal injuries.

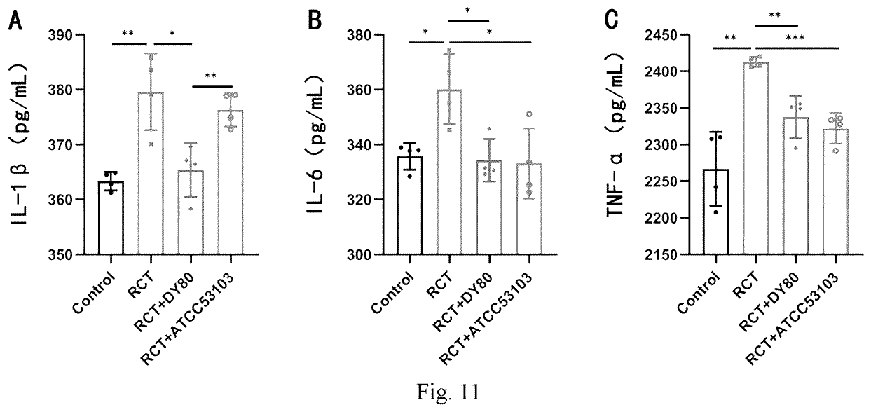

Additionally, enzyme-linked immunosorbent assay was utilized in the present example to assay the inflammatory cytokines IL-1B, IL-6 and TNF-α in the blood of the mice in each group, and the results are shown in FIG. 11. As shown in FIG. 11, the content of IL-1B, IL-6 and TNF-α in the blood of mice in the RCT group was significantly higher than that of mice in the Control group, the inflammatory cytokines of mice in the RCT+ATCC53103 group and the RCT+DY801 group were significantly reduced compared with those of mice in the RCT group, and the improvement of the inflammatory indicators of IL-1B in the mice in the RCT+DY801 group was better than that of the standard strain ATCC53103.

### Example 9 Excavation of Target Sequence of Specific Molecules of Lacticaseibacillus rhamnosus DY801

The whole genome data of other 279 strains of Lacticaseibacillus rhamnosus in the NCBI database were downloaded, and pan-genomic analyses were performed using Prokka (v1.11), Roary (v3.11.2) for the 279 strains of Lacticaseibacillus rhamnosus mentioned above and for Lacticaseibacillus rhamnosus DY801 provided by the present disclosure. After obtaining the core genes, MEGA X (v10.2.2) was utilized to identify genes with a high density of base substitutions. Based on the analytical process mentioned above, specific sequences of Lacticaseibacillus rhamnosus DY801 different from other Lacticaseibacillus rhamnosus were obtained. Primer Premier 5 was used to design primers against specific sequences to obtain a target sequence SEQ ID: 1 that specifically recognizes the specific molecule of Lacticaseibacillus rhamnosus DY801.

The validity of recognition of the target sequence by specific molecules of Lacticaseibacillus rhamnosus DY801 was verified by Polymerase Chain Reaction (PCR). The assay template was the DNA of the bacteria, and the DNA extraction method was referred to the TIANamp Bacteria DNA Kit. The amplification primers are shown in Table 5. The PCR reaction system and PCR reaction conditions of the present example were the same as the PCR reaction system and PCR reaction conditions involved in Example 1. After PCR, the product was sent to Bio-engineering company for sequencing and analyzed to be 431 bp in length and as shown in SEQ ID: 1.

TABLE 5
Amplification primer sequences

| No. | Primer sequence (5′→3′) |
|---|---|
| SEQ ID: 2 | TGATACACTCTGCGACTT |
| SEQ ID: 3 | TCACATTAGCACCGACTA |

In summary, the Lacticaseibacillus rhamnosus DY801 provided by the present disclosure may inhibit oxidative stress in the intestinal tract and damage to the small intestinal villi under radiotherapy and chemotherapy conditions by producing a large amount of glutathione in order to reduce inflammatory cytokines and accelerate the repair of intestinal tissue, having the property of tolerating artificial gastric and intestinal fluids, having a high survival rate in the stomach and intestine, and being able to colonize the intestine and restore the intestinal bacterial flora homeostasis. Additionally, compared with conventional drugs for the treatment of radiotherapy and chemotherapy intestinal injuries, the Lacticaseibacillus rhamnosus DY801 provided by the present disclosure has no obvious toxic effects on the human body, providing a high level of safety, reducing the adverse reactions caused by the drugs as well as overcoming the low therapeutic efficacy of the former. The Lacticaseibacillus rhamnosus DY801 or cultures thereof provided by the present disclosure is applied in a preparation of drugs for prevention and/or treatment of radiotherapy and chemotherapy intestinal injuries, and may be used for prevention and/or treatment of digestive disorders caused by radiotherapy and chemotherapy intestinal injuries, which solves the problems in the prior art of inability to effectively treat the radiotherapy and chemotherapy intestinal injuries occurring after radiotherapy and chemotherapy for pelvic malignant tumors, which offers great application prospects in the preparation of drugs or foodstuffs for the prevention and/or treatment of radiotherapy and chemotherapy intestinal injuries.

The above examples are only used to illustrate the technical solution of the present disclosure rather than to limit the protection scope of the present disclosure. Although the present disclosure has been described in detail with reference to the above examples, a person of ordinary skill in the art should be aware that modifications or equivalent substitutions may be carried out to the technical solution of the present disclosure, these modifications or substitutions fall within the protection scope of the present disclosure.

1. A strain of Lacticaseibacillus rhamnosus DY801, deposited in the Guangdong Microbial Culture Collection Center (GDMCC) on Oct. 9, 2022, with a deposit number of GDMCC 62853.

2. A drug for prevention and/or treatment of digestive disorders, comprising Lacticaseibacillus rhamnosus DY801 or cultures thereof, wherein the Lacticaseibacillus rhamnosus DY801 is deposited in the Guangdong Microbial Culture Collection Center (GDMCC) on Oct. 9, 2022, with a deposit number of GDMCC 62853.

3. A nucleotide sequence specifically recognizing Lacticaseibacillus rhamnosus DY801, wherein the nucleotide sequence is as shown in SEQ ID: 1, and the Lacticaseibacillus rhamnosus DY801 is deposited in the Guangdong Microbial Culture Collection Center (GDMCC) on Oct. 9, 2022, with a deposit number of GDMCC 62853.
